(19) United States
(12) Patent Application Publication
(10) Pub. No.: US 2007/0281992 A1
(43) Pub. Date: Dec. 6, 2007

(54) COMBINATION OF GYRASE B INHIBITORS AND PROTEIN SYNTHESIS INHIBITORS AND USES THEREOF

(76) Inventors: Donald Herman Batts, Kalamazoo, MI (US); Thomas Frank DeKoning, Lawton, MI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 11/695,372

(22) Filed: Apr. 2, 2007

Related U.S. Application Data
(60) Provisional application No. 60/809,778, filed on May 31, 2006.

(51) Int. Cl. A61K 31/352; A61K 31/165; A61P 31/04

(52) U.S. Cl. 514/456; 514/619

(57) ABSTRACT

The present invention relates to compounds and methods of treating, preventing, or lessening the severity of drug-resistant Gram-positive bacterial infections in mammals. The compounds consist of a combination of a gyrase B inhibitor and a protein synthesis inhibitor. These compounds demonstrate antibacterial activity against drug-resistant strains of Gram-positive bacteria, and in particular, methicillin-resistant Staphylococcus aureus (MRSA). Methods for inhibiting the activity of strains of Gram-positive bacteria and methods for treating a bacterial infection caused by such organisms are described herein.

### CROSS-REFERENCE TO RELATED APPLICATION

This application claims the benefit under 35 U.S.C. § 119(e) of U.S. Provisional Application No. 60/809,778, filed on May 31, 2006, the entire disclosure of which is hereby incorporated herein by reference.

### FIELD OF THE INVENTION

This invention is in the field of medicinal chemistry and relates to compounds and pharmaceutical compositions that treat Gram-positive infections. The pharmaceutical compounds demonstrate antibacterial activity against Gram-positive bacteria, and in particular, drug-resistant strains, such as methicillin-resistant Staphylococcus aureus (MRSA). Methods for inhibiting the activity of Gram-positive bacterial organisms and methods for treating a bacterial infection caused by such organisms are described herein.

### BACKGROUND OF THE INVENTION

The most common antimicrobial compounds are antibiotics. With the steady increase in antibiotic resistance to bacterial pathogens, there is a constant need for the development and discovery of new antibiotic compounds. Disease-causing microbes that have become resistant to antibiotic therapy are an increasing public health problem. Part of the problem is that bacteria and other microorganisms that cause infections are remarkably resilient and can develop ways to survive drugs meant to kill or weaken them. Antibiotic resistance, also known as antimicrobial resistance or drug resistance, is also aided by the well-documented increase in the use of antibiotics in many fields and applications.

The range of bacteria or other microorganisms that are affected by a certain antimicrobial compound is expressed as the spectrum of action. Antimicrobial compounds that kill or inhibit a wide range of Gram-positive and Gram-negative bacteria are said to be broad spectrum. If effective mainly against either Gram-positive or Gram-negative bacteria, they are narrow spectrum. If an antimicrobial compound is effective against a single organism or disease, it is referred to as having a limited spectrum.

“Gram-positive” bacteria are those that are stained dark blue or violet by gram staining, in contrast to gram-negative bacteria, which are not affected by the stain. The stain is caused by a high amount of peptidoglycan in the cell wall, which typically, but not always, lacks the secondary membrane and lipopolysaccharide layer found in Gram-negative bacteria.

Peptidoglycan, also known as murein, is a substance that forms a homogenous layer lying outside the plasma membrane in prokaryotes. It serves a structural role in bacterial cell walls giving bacteria shape, strength, and counteracting the osmotic pressure of the cytoplasm. It is also involved in binary fission of the bacterial cell. The formation of the peptidoglycan layer in bacteria, specifically the crosslinking enzyme transpeptidase, is the target for drugs, such as penicillin. The peptidoglycan layer is thicker in Gram-positive bacteria (20 to 80 nm) than in Gram-negative bacteria (7 to 8 nm). It forms around 90 percent and 10 percent of dry weight of Gram-positive and Gram-negative bacteria, respectively.

Bacterial resistance to antibiotics has long been recognized. As a result of resistance, some bacterial infections are either difficult to treat with antibiotics or even untreatable. This problem has become especially serious with the recent development of some level of drug resistance in certain strains of Gram-positive bacteria, such as Staphylococcus aureus (SA), Streptococcus pneumoniae (SP), and Enterococcus. There is the fear that the genes which induce resistance might spread to more deadly organisms, such as Staphylococcus aureus, where methicillin resistance (MRSA) is already prevalent. MRSA may also be known as oxacillin-resistant Staphylococcus aureus (ORSA) and multi-resistant Staphylococcus aureus.

Staphylococcus aureus, often referred to simply as “staph,” are bacteria commonly carried on the skin or in the nose of healthy people. Approximately 25 percent to 30 percent of the population is colonized (when bacteria are present, but not causing an infection) in the nose with staph bacteria. Sometimes, staph can cause an infection. Staph bacteria are one of the most common causes of skin infections in the United States. Most of these skin infections are minor (such as pimples and boils) and can be treated without antibiotics (also known as antimicrobials or antibacterials). However, staph bacteria also can cause serious infections (such as surgical wound infections, bloodstream infections, and pneumonia).

Some staph bacteria are resistant to antibiotics. MRSA is a type of staph that is resistant to antibiotics called beta-lactams. Beta-lactam antibiotics include methicillin and other more common antibiotics, such as oxacillin, penicillin, and amoxicillin. While 25 percent to 30 percent of the population is colonized with staph, approximately one percent is colonized with MRSA.

Staph infections, including MRSA, occur most frequently among persons in hospitals and healthcare facilities (such as nursing homes and dialysis centers) who have weakened immune systems. These healthcare-associated staph infections include surgical wound infections, urinary tract infections, bloodstream infections, and pneumonia.

Staph and MRSA can also cause illness in persons outside of hospitals and healthcare facilities. MRSA infections that are acquired by persons who have not been recently (within the past year) hospitalized or had a medical procedure (such as dialysis, surgery, or catheters) are known as community-associated (CA-MRSA) infections. Staph or MRSA infections in the community are usually manifested as skin infections, such as pimples and boils, and occur in otherwise healthy people.

As a result of the need to combat drug-resistant bacteria and the increasing failure of the available drugs, there has been a resurgent interest in discovering new antibiotics. One attractive strategy for developing new antibiotics is to inhibit DNA gyrase, a bacterial enzyme necessary for DNA replication, and therefore, necessary for bacterial cell growth and division. Gyrase activity is also associated with events in DNA transcription, repair, and recombination.

Gyrase is one of the topoisomerases, a class of enzymes that alter the supercoiling of double-stranded DNA. The topoisomerases act by transiently cutting one or both strands of the DNA. Topoisomerase type I cuts one strand, whereas topoisomerases type II cuts both strands of the DNA to relax the coil and extend the DNA molecule. The regulation of DNA supercoiling is essential to DNA transcription and replication, when the DNA helix must unwind to permit the proper function of the enzymatic machinery involved in these processes. Topoisomerases serve to maintain both the transcription and replication of DNA.

Gyrase is an enzyme in the topoisomerase family that passes one double strand of DNA through another double strand of DNA. Because gyrase changes the linking number of the DNA by two in each enzymatic step, it is classified as a type II topoisomerase. Gyrase has two main activities: introducing negative supercoils and relaxing positive supercoils. The unique ability of gyrase to introduce negative supercoils into DNA is what allows bacterial DNA, but not eukaryotic DNA, to have free negative supercoils. Gyrase is only found in bacteria; it is not found in eukaryotes, a property that makes gyrase a good target for antibiotics. Two classes of antibiotics that inhibit gyrase are the coumarins, including novobiocin, and the quinolones, which include naladixic acid and ciprofloxacin (better known as Cipro). The ability of gyrase to relax positive supercoils comes into play during DNA replication. The right-handed nature of the DNA double helix causes positive supercoils to accumulate ahead of a translocating enzyme, in the case of DNA replication, a DNA polymerase. The ability of gyrase (and topoisomerase IV) to relax positive supercoils allows superhelical tension ahead of the polymerase to be released so that replication can continue.

Specifically, gyrase itself controls DNA supercoiling and relieves topological stress that occurs when the DNA strands of a parental duplex are untwisted during the replication process. Gyrase also catalyzes the conversion of relaxed, closed circular duplex DNA to a negatively superhelical form, which is more favorable for recombination. The mechanism of the supercoiling reaction involves the wrapping of gyrase around a region of the DNA, double strand breaking in that region, passing a second region of the DNA through the break, and rejoining the broken strands. Such a cleavage mechanism is characteristic of a type II topoisomerase. The supercoiling reaction is driven by the binding of ATP to gyrase. The ATP is then hydrolyzed during the reaction. This ATP binding and subsequent hydrolysis cause conformational changes in the DNA-bound gyrase that are necessary for its activity. It has also been found that the level of DNA supercoiling (or relaxation) is dependent on the ATP/ADP ratio. In the absence of ATP, gyrase is only capable of relaxing supercoiled DNA.

Bacterial DNA gyrase is a 400 kilodalton protein tetramer consisting of two A (GyrA) and two B subunits (GyrB). Binding and cleavage of the DNA is associated with GyrA (gyrase A), whereas ATP is bound and hydrolyzed by the GyrB (gyrase B) protein. GyrB consists of an amino-terminal domain which has the ATPase activity, and a carboxy-terminal domain which interacts with GyrA and DNA. By contrast, eukaryotic type II topoisomerases are homodimers that can relax negative and positive supercoils, but cannot introduce negative supercoils. Ideally, an antibiotic based on the inhibition of bacterial DNA gyrase would be selective for this enzyme and be relatively inactive against the eukaryotic type II topoisomerases.

As referenced above, the widely used quinolone antibiotics inhibit bacterial DNA gyrase. Examples of the quinolones include the early compounds such as nalidixic acid and oxolinic acid, as well as the later, more potent fluoroquinolones such as norfloxacin, ciprofloxacin, and trovafloxacin. These compounds bind to GyrA and stabilize the cleaved complex, thus inhibiting overall gyrase function, leading to cell death. However, drug resistance has also been recognized as a problem for this class of compounds (WHO Report, “Use of Quinolones in Food Animals and Potential Impact on Human Health,” 1998). With the quinolones, as with other classes of antibiotics, bacteria exposed to earlier compounds often quickly develop cross-resistance to more potent compounds in the same class.

There are fewer known inhibitors that bind to gyrase B. Examples include the coumarins, novobiocin and coumermycin A1, cyclothialidine, cinodine, and clerocidin. The coumarins have been shown to bind to gyrase B very tightly. For example, novobiocin makes a network of hydrogen bonds with the protein and several hydrophobic contacts. Despite being potent inhibitors of gyrase supercoiling in bacteria, the coumarins have not been widely used as antibiotics due to low permeability in bacteria, eukaryotic toxicity, and somewhat poor water solubility. As bacterial resistance to antibiotics has become an important public health problem, there is an even greater need to develop better antibiotics. More particularly, there is a need for antibiotics that represent new methods of treating bacterial infections.

### SUMMARY OF THE INVENTION

It has been found that the compounds of this invention are useful in methods of treating, preventing, or lessening the severity of a Gram-positive bacterial infection. The combination of a gyrase B inhibitor and a protein synthesis inhibitor is used to treat Gram-positive infections. This combination retards the development of resistance to both agents through the combination of both agents. In addition, the combination suppresses toxin production in Gram-positive infections, where the infections are of clinical significance (such as Pantin Valentine Leukocidin, anti-DNAase, anti-streptokinase, Enterotoxin B and C, and the toxins associated with necrotizing fasciitis caused by both Staphylococci and Streptococci). The combination also reduces inflammation caused by Gram-positive bacterial infections.

These and other features, advantages and objects of the present invention will be further understood and appreciated by those skilled in the art by reference to the following specification.

### BRIEF DESCRIPTION OF THE DRAWINGS

FIG. 1 illustrates the gyrase B protein with the overlap of the novobiocin, shown in white, and the ADPNP binding sites; and

FIG. 2 illustrates the gyrase B protein with the overlap of the cyclothialidine, shown in white, and the ADPNP binding sites.

### DETAILED DESCRIPTION OF THE PREFERRED EMBODIMENTS

In a first aspect, the invention comprises applying the combination of a gyrase B inhibitor and a protein synthesis inhibitor to a Gram-positive strain of bacteria. The method demonstrates effectiveness for inhibiting the bacterial activity of Gram-positive bacteria, and in particular, resistant strains, such as MRSA. In this aspect of the invention, the compound can be applied in any suitable manner for commingling the combination with the bacteria.

“Gram-positive bacteria” include cocci, nonsporulating rods, and sporulating rods. The genera of Gram-positive bacteria include, for example, Actinomyces, Bacillus, Clostridium, Corynebacterium, Erysipelothrix, Lactobacillus, Listeria, Mycobacterium, Myxococcus, Nocardia, Staphylococcus, and Streptococcus. The Gram-positive bacterium can be a Bacillus, Enterococcus, Mycobacterium, Staphylococcus, or Streptococcus strain.

DNA topoisomerases are enzymes that control and modify the topological states of DNA in cells. Bacterial DNA gyrase is a type II DNA topoisomerase which catalyses the negative supercoiling of prokaryotic DNA, utilizing the free energy released by the hydrolysis of ATP. DNA gyrase is the target of two classes of antibiotic drugs: the quinolones and the coumarins. DNA gyrase consists of two proteins (A and B), with the active species being a heterotetramer (A2B2). Topoisomerase IV is another type II enzyme found in bacteria. Unlike DNA gyrase, it is unable to catalyze the supercoiling of DNA, merely its relaxation. It appears the role of this enzyme, in vitro, is to decatenate the daughter chromosomes in the final stages of DNA replication. The enzyme shows a great deal of sequence homology with DNA gyrase. The enzyme comprises two subunits, ParC and ParE. The ParC protein is homologous to the gyrase A protein, while the ParE subunit is homologous to the gyrase B protein.

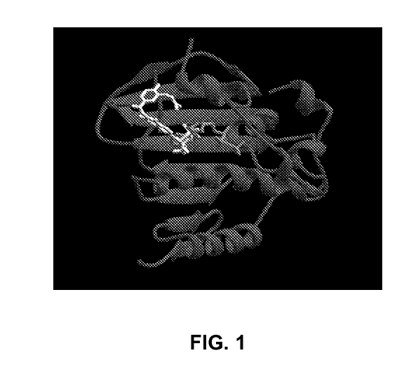

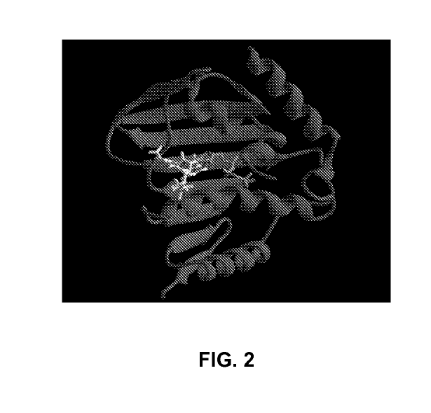

The gyrase B protein is the target of the coumarin antibiotics. The structures of the complexes of the 24 kDa N-terminal domain of the B protein with novobiocin and chlorobiocin at 2.7 Å and 1.9 Å, respectively, are shown in FIG. 1. The gyrase B protein is also inhibited by a group of naturally occurring cyclic peptides called cyclothialidines. The structure of the 24 kDa B protein fragment complexed with a cyclothialidine at 2 Å resolution is shown in FIG. 2.

Another class of small molecule natural products that are useful as antibiotics are protein synthesis inhibitors. These protein synthesis inhibitors include macrocyclic lactones. This group of compounds shares the presence of a large lactone ring with various ring substituents. They can be further classified into subgroups, depending on the ring size and other characteristics. The macrolides, for example, contain 12-, 14-, 16-, or 17-membered lactone rings glycosidically linked to one or more aminosugars and/or deoxysugars. They are inhibitors of protein synthesis, and are particularly effective against Gram-positive bacteria. Erythromycin A, a well-studied macrolide produced by Saccharopolyspora erythraea, consists of a 14-membered lactone ring linked to two deoxysugars.

Still another class of molecules that are protein synthesis inhibitors are derivatives of quinones. Quinones are aromatic compounds with two carbonyl groups on a fully unsaturated ring. The compounds can be broadly classified into subgroups according to the number of aromatic rings present, i.e., benzoquinones, napthoquinones, etc. A well studied group is the tetracyclines, which contain a napthacene ring with different substituents. Tetracyclines are protein synthesis inhibitors and are effective against both Gram-positive and Gram-negative bacteria, as well as rickettsias, mycoplasma, and spirochetes.

Protein synthesis inhibitors act by inhibiting translation at the level of the ribosome, binding the 30S and/or 50S subunits of the ribosomes, which provides the selective toxicity desired for an antimicrobial drug. The most important antibiotics with this mode of action are the tetracyclines, chloramphenicol, the macrolides (e.g., erythromycin), lincosamides (e.g., clindamycin), oxazolidinones (e.g., linezolid), fusidic acid, mupirocin (e.g., Bactroban), and the aminoglycosides (e.g., gentamicin or streptomycin).

The aminoglycosides are products of Streptomyces species and include streptomycin, kanamycin, tobramycin, and gentamicin. These antibiotics exert their activity by binding to bacterial ribosomes and preventing the initiation of protein synthesis. Aminoglycosides have been used against a wide variety of bacterial infections caused by Gram-positive and Gram-negative bacteria.

The tetracyclines are antibiotics which are natural products of Streptomyces, although some are produced semisynthetically. Tetracycline, chlortetracycline, and doxycycline are the best known. The tetracyclines are broad spectrum antibiotics with a wide range of activity against both Gram-positive and Gram-negative bacteria. The tetracyclines act by blocking the binding of aminoacyl tRNA to the A site on the ribosome. Tetracyclines inhibit protein synthesis on isolated 70S or 80S (eukaryotic) ribosomes, and in both cases, their effect is on the small ribosomal subunit. However, most bacteria possess an active transport system for tetracycline that will allow intracellular accumulation of the antibiotic at concentrations 50 times as great as that in the medium. This greatly enhances its antibacterial effectiveness and accounts for its specificity of action, since an effective concentration cannot be accumulated in animal cells.

Chloramphenicol, originally purified from the fermentation of a Streptomyces, currently is produced by chemical synthesis. Chloramphenicol inhibits the bacterial enzyme peptidyl transferase thereby preventing the growth of the polypeptide chain during protein synthesis. Chloramphenicol is entirely selective for 70S ribosomes and does not affect 80S ribosomes.

The macrolides and lincosamides, such as erythromycin and clindamycin, inhibit bacterial protein synthesis by binding to the 50S ribosomal subunit.

The oxazolidinones bind to the 23s ribosomal RNA subunits of the 50s ribosome and inhibit initiation of protein synthesis by not allowing the first peptide bond to form between f-met transfer RNA and the first amino acid.

Fusidic acid exerts its mode of action by inhibition of protein synthesis by the prevention of translocation on the ribosome.

Mupirocin apparently exerts its antimicrobial activity by reversibly inhibiting isoleucyl-transfer RNA, thereby inhibiting bacterial protein and RNA synthesis.

The combination of a gyrase B inhibitor and a protein synthesis inhibitor is used to treat Gram-positive bacterial infections. The gyrase B inhibitor can be a drug like novobiocin. The dose of novobiocin is in the preferred range of 100 mg to 500 mg per dose. The protein synthesis inhibitor can be a tetracycline (such as tetracycline HCl, doxycycline, or minocycline) in the preferred range of 50 mg to 500 mg given one to three times a day. Further protein synthesis inhibitors may include erythromycin, clindamycin, lincomycin, clarithromycin, azithromycin, oleandomycin, picromycin, narbomycin, linezolid, fusidic acid, mupirocin, gentamicin, thiamphenicol, and chloramphenicol. The combination gives better results in vitro against Gram-positive organisms (e.g., Staphylococci and Streptococci) than either drug alone, and development of resistance should be delayed by the combination, as opposed to the single agents.

An oral antibiotic combination therapy containing both novobiocin and tetracycline was marketed by The Upjohn Company from 1957 to early in 1970. Marketed under the trade name Panalba™, this dual therapy provided physicians with an oral option for treating antibiotic-resistant staphylococci. Panalba™ was withdrawn from the market because of concerns that the combination of novobiocin and tetracycline could lead to increased drug resistance in bacteria. In addition, the FDA believed that tetracycline was more effective than Panalba™, when used alone. Panalba™ was developed and launched prior to the discovery of MRSA in the United Kingdom in 1961. Panalba™ was off the market before the discovery of CA-MRSA in the early 1980s.

The data below describes the activity of novobiocin/tetracycline (in comparison to that of other agents) against recent clinical isolates of methicillin-sensitive (MSSA), methicillin-resistant (MRSA), vancomycin-intermediate (VISA), and linezolid-resistant (LRSA) staphylococci.

Materials and Methods

Antibiotics: novobiocin, tetracycline, minocycline, oxacillin, and vancomycin were all purchased from Sigma-Aldrich. Linezolid was obtained from Pfizer, Inc. Novobiocin/tetracycline and novobiocin/minocycline were tested in a 2.5:1 ratio to mimic the serum levels. (Vavra, J. J., 1967, Development of resistance to novobiocin, tetracycline, and novobiocin-tetracycline combinations in Staphylococcus aureus populations, J. Bacteriol 93(3):801). Compounds were tested across a concentration range consisting of 11 two-fold serial dilutions of test agent.

Stock solutions were prepared in sterile deionized water; novobiocin stock concentration was 1600 μg/mL and all other drugs stock solutions were prepared at 640 μg/mL. The stock solutions were allowed to sit at room temperature for one hour to auto-sterilize prior to use in the test. All compounds were in solution in water. The control agent was vancomycin hydrochloride, Sigma Chemical Lot # 015K0825.

The test organisms for the assay were 52 MSSA, 52 MRSA, 8 VISA, 7 VISE, and 3 LRSA isolates. Quality control strains included Staphylococcus aureus 0100 (ATCC 29213) and Enterococcus faecalis (ATCC 29212).

The MIC assay was performed according to published NCCLS guidelines. (National Committee for Clinical Laboratory Standards, Methods for Dilution Antimicrobial Susceptibility Tests for Bacteria that Grow Aerobically; Approved Standard-Sixth Edition, NCCLS Document M7-A6 [ISBN 1-56238-486-4], NCCLS, 940 West Valley Road, Suite 1400, Wayne, Pa. 19087-1898 USA, 2003). The test medium was Mueller Hinton II Broth for all organisms.

Results

The line listing of MIC values for all agents is included in Appendix A, and quality control results for all assays are presented in Appendix B. The results of the assays are summarized in Table 1. Examination of the data for MSSA in Table 1 reveals that the MlC₉₀ value for the novobiocin/tetracycline combination at a fixed concentration of 2.5:1 was 0.312/0.12 μg/mL, indicating that this antibiotic combination provided good activity against recent MSSA isolates. This activity was maintained with MRSA as well (MlC₉₀=0.15/0.06 μg/mL).

Appendix A reveals that MSSA isolate #1728 was resistant to novobiocin alone, producing an MIC value of 10 μg/mL as opposed to the other 51 MSSA isolates exhibiting a novobiocin MIC range of 0.08-0.62 μg/mL. It is interesting to note that the novobiocin/tetracycline combination produced an MIC of 0.62/0.25 μg/mL for isolate #1728, indicating that the addition of tetracycline provided coverage for novobiocin resistance.

S. aureus isolates are susceptible to tetracycline at <4 μg/mL, have intermediate resistance at 8 μg/mL, and are fully resistant at >16 μg/mL. Examination of Appendix A shows that tetracycline resistance was noted in MSSA isolates 784 and 999; MRSA isolates 757, 769, 1004, 1729, 2009, and 2011; VISA isolates 2014 and 2019; VISE isolate 2020; and LRSA isolate 2025. Of particular interest to the present study is that the novobiocin/tetracycline combination was active against all of these tetracycline-resistant MSSA and MRSA, providing MIC values very similar to tetracycline-sensitive staphylococci. Indeed, it appears that the combination of novobiocin and tetracycline provides excellent activity against MSSA, MRSA, VISA, VISE, and LRSA isolates—including those resistant to either novobiocin or tetracycline.

TABLE 1
MIC Range, MIC_{50,}, and MIC₉₀ Values for Methicillin-Susceptible Staphylococcus aureus Isolates

| Organism | | | | | | | | | | | | | | | | | |
|---|---|---|---|---|---|---|---|---|---|---|---|---|---|---|---|---|---|
| Phenotype | | | | | | | | | | | | | | | | | |
| (No. | | | | | | | | | | | | | | | MIC Range | MIC₅₀ | MIC₉₀ |
| Tested) | Drug¹ | No. Strains Inhibited at Concentration (μg/ml) | | | | | | | | | | | | | (μg/ml) | (μg/ml) | (μg/ml) |
| MSSA² | NB/TE | ≦.04/ | .08/ | .15/ | .312/ | .625/ | 1.25/ | 2.5/1 | 5/2 | 10/4 | 20/8 | 40/ | | | | | |
| (52) | | .015 | .03 | .06 | .12 | .25 | .5 | | | | | 16 | | | | | |
| | | | 4 | 36 | 11 | 1 | | | | | | | | | .08/.03-.625/ | .15/.06 | .312/.12 |
| | | | | | | | | | | | | | | | .25 | | |
| | NB/MI | ≦.04/ | .08/ | .15/ | .312/ | .625/ | 1.25/ | 2.5/1 | 5/2 | 10/4 | 20/8 | 40/ | | | | | |
| | | .015 | .03 | .06 | .12 | .25 | .5 | | | | | 16 | | | | | |
| | | 1 | 18 | 33 | | | | | | | | | | | ≦.04/.015-.15/ | .15/.06 | .15/.06 |
| | | | | | | | | | | | | | | | .06 | | |
| | NB | ≦.04 | .08 | .15 | .312 | .62 | 1.25 | 2.5 | 5 | 10 | 20 | 40 | | | | | |
| | | | 3 | 24 | 23 | 1 | | | | 1 | | | | | .08-10 | .15 | .312 |
| | TE | ≦.015 | .03 | .06 | .12 | .25 | .5 | 1 | 2 | 4 | 8 | 16 | >16 | | | | |
| | | | | | | 2 | 45 | 3 | | | | 1 | 1 | | .25->16 | .5 | .5 |
| | MI | ≦.015 | .03 | .06 | .12 | .25 | .5 | 1 | 2 | 4 | 8 | 16 | >16 | | | | |
| | | | | 19 | 33 | | | | | | | | | | .06-.12 | .12 | .12 |
| | LZD | ≦.03 | .03 | .06 | .12 | .25 | .5 | 1 | 2 | 4 | 8 | 16 | 32 | >32 | | | |
| | | | | | | | | 1 | 36 | 15 | | | | | 1-4 | 2 | 4 |
| | VA | ≦.03 | .03 | .06 | .12 | .25 | .5 | 1 | 2 | 4 | 8 | 16 | 32 | >32 | | | |
| | | | | | | | 31 | 21 | | | | | | | .5-1 | .5 | 1 |
| | OX | ≦.03 | .03 | .06 | .12 | .25 | .5 | 1 | 2 | 4 | 8 | 16 | 32 | >32 | | | |
| | | | | | 8 | 40 | 4 | | | | | | | | .12-.5 | .25 | .25 |

¹Drugs: NB = novobiocin, TE = tetracycline, MI = minocycline, LZD = linezolid, VA—vancomycin, OX = oxacillin
²MSSA = methicillin-susceptible Staphylococcus aureus

MIC Range, MIC_{50,}, and MIC₉₀ Values for Methicillin-Resistant Staphylococcus aureus Isolates

| Organism | | | | | | | | | | | | | | | | | |
|---|---|---|---|---|---|---|---|---|---|---|---|---|---|---|---|---|---|
| Phenotype | | | | | | | | | | | | | | | | | |
| (No. | | | | | | | | | | | | | | | MIC Range | MIC₅₀ | MIC₉₀ |
| Tested) | Drug¹ | No. Strains Inhibited at Concentration (μg/ml) | | | | | | | | | | | | | (μg/ml) | (μg/ml) | (μg/ml) |
| MRSA² | NB/TE | ≦.04/ | .08/ | .15/ | .312/ | .625/ | 1.25/ | 2.5/1 | 5/2 | 10/4 | 20/8 | 40/ | | | | | |
| (52) | | .015 | .03 | .06 | .12 | .25 | .5 | | | | | 16 | | | | | |
| | | 7 | 13 | 29 | 3 | | | | | | | | | | ≦.04/.015-.312/ | .15/.06 | .15/.06 |
| | | | | | | | | | | | | | | | .12 | | |
| | NB/MI | ≦.04/ | .08/ | .15/ | .312/ | .625/ | 1.25/ | 2.5/1 | 5/2 | 10/4 | 20/8 | 40/ | | | | | |
| | | .015 | .03 | .06 | .12 | .25 | .5 | | | | | 16 | | | | | |
| | | 4 | 14 | 34 | | | | | | | | | | | ≦.04/.015-.15/ | .15/.06 | .15/.06 |
| | | | | | | | | | | | | | | | .06 | | |
| | NB | ≦.04 | .08 | .15 | .312 | .62 | 1.25 | 2.5 | 5 | 10 | 20 | 40 | | | | | |
| | | 6 | 13 | 21 | 11 | 1 | | | | | | | | | ≦.04-.62 | .15 | .312 |
| | TE | ≦.015 | .03 | .06 | .12 | .25 | .5 | 1 | 2 | 4 | 8 | 16 | >16 | | | | |
| | | | | | | 3 | 36 | 6 | | 1 | | 1 | 5 | | .25->16 | .5 | 16 |
| | MI | ≦.015 | .03 | .06 | .12 | .25 | .5 | 1 | 2 | 4 | 8 | 16 | >16 | | | | |
| | | | | 7 | 42 | | | 1 | | | 1 | 1 | | | .06-.16 | .12 | .12 |
| | LZD | ≦.03 | .03 | .06 | .12 | .25 | .5 | 1 | 2 | 4 | 8 | 16 | 32 | >32 | | | |
| | | | | | | | | | 35 | 17 | | | | | 2-4 | 2 | 4 |
| | VA | ≦.03 | .03 | .06 | .12 | .25 | .5 | 1 | 2 | 4 | 8 | 16 | 32 | >32 | | | |
| | | | | | | | 27 | 23 | 2 | | | | | | .5-2 | .5 | 1 |
| | OX | ≦.03 | .03 | .06 | .12 | .25 | .5 | 1 | 2 | 4 | 8 | 16 | 32 | >32 | | | |
| | | | | | | | | | | 1 | 6 | 9 | 17 | 19 | 4->32 | 32 | >32 |

¹Drugs: NB—novobiocin, TE—tetracycline, MI = minocycline, LZD = linezolid, VA = vancomycin, OX = oxacillin
²MRSA = methicillin-resistant Staphylococcus aureus

MIC Range, MIC_{50,}, and MIC₉₀ Values for Vancomycin-intermediate Staphylococcus aureus Isolates

| Organism | | | | | | | | | | | | | | | | | |
|---|---|---|---|---|---|---|---|---|---|---|---|---|---|---|---|---|---|
| Phenotype | | | | | | | | | | | | | | | MIC Range | MIC₅₀ | MIC₉₀ |
| (No. Tested) | Drug¹ | | | | | | | | | | | | | | (μg/ml) | (μg/ml) | (μg/ml) |
| VISA² (2) | NB/TE | ≦.04/ | .08/ | .15/ | .312/ | .625/ | 1.25/ | 2.5/1 | 5/2 | 10/4 | 20/8 | 40/ | | | | | |
| | | .015 | .03 | .06 | .12 | .25 | .5 | | | | | 16 | | | | | |
| | | 1 | 1 | | | | | | | | | | | | ≦.04/.015-.08/ | ≦.04/ | |
| | | | | | | | | | | | | | | | .03 | .015 | |
| | NB/MI | ≦.04/ | .08/ | .15/ | .312/ | .625/ | 1.25/ | 2.5/1 | 5/2 | 10/4 | 20/8 | 40/ | | | | | |
| | | .015 | .03 | .06 | .12 | .25 | .5 | | | | | 16 | | | | | |
| | | 1 | 1 | | | | | | | | | | | | ≦.04/.015-.08/ | ≦.04/ | |
| | | | | | | | | | | | | | | | .03 | .015 | |
| | NB | ≦.04 | .08 | .15 | .312 | .62 | 1.25 | 2.5 | 5 | 10 | 20 | 40 | | | | | |
| | | 1 | 1 | | | | | | | | | | | | ≦.04-.08 | ≦.04 | |
| | TE | ≦.015 | .03 | .06 | .12 | .25 | .5 | 1 | 2 | 4 | 8 | 16 | >16 | | | | |
| | | | | | | 1 | | | 1 | | | | | | .25-2 | .25 | |
| | MI | ≦.015 | .03 | .06 | .12 | .25 | .5 | 1 | 2 | 4 | 8 | 16 | >16 | | | | |
| | | | | 1 | | 1 | | | | | | | | | .06-25 | .06 | |
| | LZD | ≦.03 | .03 | .06 | .12 | .25 | .5 | 1 | 2 | 4 | 8 | 16 | 32 | >32 | | | |
| | | | | | | | | 1 | 1 | | | | | | 1-2 | 1 | |
| | VA | ≦.03 | .03 | .06 | .12 | .25 | .5 | 1 | 2 | 4 | 8 | 16 | 32 | >32 | | | |
| | | | | | | | | | | | 2 | | | | 8 | 8 | |
| | OX | ≦.03 | .03 | .06 | .12 | .25 | .5 | 1 | 2 | 4 | 8 | 16 | 32 | >32 | | | |
| | | | | | | | | | | | | | | 2 | >32 | >32 | |

¹Drugs: NB = novobiocin, TE = tetracycline, MI = minocycline, LZD—linezolid, VA—vancomycin, OX = oxacillin
²VISA—vancomycin-intermediate Staphylococcus aureus

MIC Range, MIC_{50,}, and MIC₉₀ Values for Unconfirmed²
Vancomycin-intermediate Staphylococcus aureus Isolates

| Organism | | | | | | | | | | | | | | | | | |
|---|---|---|---|---|---|---|---|---|---|---|---|---|---|---|---|---|---|
| Phenotype | | | | | | | | | | | | | | | MIC Range | MIC₅₀ | MIC₉₀ |
| (No. Tested) | Drug¹ | | | | | | | | | | | | | | (μg/ml) | (μg/ml) | (μg/ml) |
| VISA² (6) | NB/TE | ≦0.4/ | .08/ | .15/ | .312/ | .625/ | 1.25/ | 2.5/1 | 5/2 | 10/4 | 20/8 | 40/ | | | | | |
| unconfirmed | | .015 | .03 | .06 | .12 | .25 | .5 | | | | | 16 | | | | | |
| | | 3 | | 3 | | | | | | | | | | | ≦.04/.015-.15/ | ≦.04/ | |
| | | | | | | | | | | | | | | | .06 | .015 | |
| | NB/MI | ≦0.4/ | .08/ | .15/ | .312/ | .625/ | 1.25/ | 2.5/1 | 5/2 | 10/4 | 20/8 | 40/ | | | | | |
| | | .015 | .03 | .06 | .12 | .25 | .5 | | | | | 16 | | | | | |
| | | 2 | 3 | 1 | | | | | | | | | | | ≦.04/.015-.15/ | .08/.03 | |
| | | | | | | | | | | | | | | | .06 | | |
| | NB | ≦0.4 | .08 | .15 | .312 | .62 | 1.25 | 2.5 | 5 | 10 | 20 | 40 | | | | | |
| | | 3 | 1 | 2 | | | | | | | | | | | ≦.04-.15 | ≦.04 | |
| | TE | ≦.015 | .03 | .06 | .12 | .25 | .5 | 1 | 2 | 4 | 8 | 16 | >16 | | | | |
| | | | | 1 | | 1 | 2 | | | | | | 2 | | 0.6->16 | 0.5 | |
| | MI | ≦.015 | .03 | .06 | .12 | .25 | .5 | 1 | 2 | 4 | 8 | 16 | >16 | | | | |
| | | 1 | 1 | 1 | 1 | | | | 2 | | | | | | ≦.015-2 | .06 | |
| | LZD | ≦.03 | .03 | .06 | .12 | .25 | .5 | 1 | 2 | 4 | 8 | 16 | 32 | >32 | | | |
| | | | | | | | | 2 | 3 | 1 | | | | | 1-4 | 2 | |
| | VA | ≦.03 | .03 | .06 | .12 | .25 | .5 | 1 | 2 | 4 | 8 | 16 | 32 | >32 | | | |
| | | | | | | | | | | 6 | | | | | 4 | 4 | |
| | OX | ≦.03 | .03 | .06 | .12 | .25 | .5 | 1 | 2 | 4 | 8 | 16 | 32 | >32 | | | |
| | | | | | | 1 | | | | 2 | | | | 3 | .25->32 | 4 | |

¹Drugs: NB = novobiocin, TE—tetracycline, MI—minocycline, LZD—linezolid, VA—vancomycin, OX—oxacillin
²VISA = VISA should have a vancomycin MIC of 8 ug/ml: These isolates tested at 4 ug/ml, and therefore are unconfirmed vancomycin-intermediate
Staphylococcus aureus

MIC Range, MIC_{50,}, and MIC₉₀ Values for Vancomycin-intermediate Staphylococcus epidermidis Isolates

| Organism | | | | | | | | | | | | | | | | | |
|---|---|---|---|---|---|---|---|---|---|---|---|---|---|---|---|---|---|
| Phenotype | | | | | | | | | | | | | | | MIC Range | MIC₅₀ | MIC₉₀ |
| (No. Tested) | Drug¹ | | | | | | | | | | | | | | (μg/ml) | (μg/ml) | (μg/ml) |
| VISE² (4) | NB/TE | ≦0.4/ | .08/ | .15/ | .312/ | .625/ | 1.25/ | 2.5/1 | 5/2 | 10/4 | 20/8 | 40/ | | | | | |
| | | .015 | .03 | .06 | .12 | .25 | .5 | | | | | 16 | | | | | |
| | | 3 | 1 | | | | | | | | | | | | ≦.04/.015-.08/ | ≦.04/ | |
| | | | | | | | | | | | | | | | .03 | .015 | |
| | NB/MI | ≦.04/ | .08/ | .15/ | .312/ | .625/ | 1.25/ | 2.5/1 | 5/2 | 10/4 | 20/8 | 40/ | | | | | |
| | | .015 | .03 | .06 | .12 | .25 | .5 | | | | | 16 | | | | | |
| | | 1 | 3 | | | | | | | | | | | | ≦.04/.015-.08/ | .08/.03 | |
| | | | | | | | | | | | | | | | .03 | | |
| | NB | ≦0.4 | .08 | .15 | .312 | .62 | 1.25 | 2.5 | 5 | 10 | 20 | 40 | | | | | |
| | | 3 | 1 | | | | | | | | | | | | ≦.04-.08 | ≦.04 | |
| | TE | ≦.015 | .03 | .06 | .12 | .25 | .5 | 1 | 2 | 4 | 8 | 16 | >16 | | | | |
| | | | | | | 1 | 1 | | 1 | | | | 1 | | 0.25->16 | 0.5 | |
| | MI | ≦.015 | .03 | .06 | .12 | .25 | .5 | 1 | 2 | 4 | 8 | 16 | >16 | | | | |
| | | | | 2 | 1 | 1 | | | | | | | | | .06-.25 | .06 | |
| | LZD | | .03 | .06 | .12 | .25 | .5 | 1 | 2 | 4 | 8 | 16 | 32 | >32 | | | |
| | | | | | | | | 1 | 3 | | | | | | 1-2 | 2 | |
| | VA | | .03 | .06 | .12 | .25 | .5 | 1 | 2 | 4 | 8 | 16 | 32 | >32 | | | |
| | | | | | | | | | | | 4 | | | | 8 | 8 | |
| | OX | | .03 | .06 | .12 | .25 | .5 | 1 | 2 | 4 | 8 | 16 | 32 | >32 | | | |
| | | | | | | | 1 | | 1 | | 1 | 1 | | | 0.5-16 | 2 | |

¹Drugs: NB = novobiocin, TE = tetracycline, MI = minocycline, LZD = linezolid, VA = vancomycin, OX = oxacillin
²VISE = vancomycin-intermediate Staphylococcus epidermidis

MIC Range, MIC_{50,}, and MIC₉₀ Values for Unconfirmed² Vancomycin-intermediate
Staphylococcus epidermidis Isolates

| Organism | | | | | | | | | | | | | | | | | |
|---|---|---|---|---|---|---|---|---|---|---|---|---|---|---|---|---|---|
| Phenotype | | | | | | | | | | | | | | | MIC Range | MIC₅₀ | MIC₉₀ |
| (No. Tested) | Drug¹ | | | | | | | | | | | | | | (μg/ml) | (μg/ml) | (μg/ml) |
| VISE² (3) | NB/TE | ≦.04/ | .08/ | .15/ | .312/ | .625/ | 1.25/ | 2.5/1 | 5/2 | 10/4 | 20/8 | 40/ | | | | | |
| unconfirmed | | .015 | .03 | .06 | .12 | .25 | .5 | | | | | 16 | | | | | |
| | | 1 | | | 1 | | | | 1 | | | | | | ≦.04/.015-5/2 | .31/.12 | |
| | NB/MI | ≦.04/ | .08/ | .15/ | .312/ | .625/ | 1.25/ | 2.5/1 | 5/2 | 10/4 | 20/8 | 40/ | | | | | |
| | | .015 | .03 | .06 | .12 | .25 | .5 | | | | | 16 | | | | | |
| | | | 1 | 1 | | 1 | | | | | | | | | .08/.03-.625/ | .15/.06 | |
| | | | | | | | | | | | | | | | .25 | | |
| | NB | ≦0.4 | .08 | .15 | .312 | .62 | 1.25 | 2.5 | 5 | 10 | 20 | 40 | | | | | |
| | | 1 | | | 1 | | | | 1 | | | | | | ≦.04-5 | .312 | |
| | TE | ≦.015 | .03 | .06 | .12 | .25 | .5 | 1 | 2 | 4 | 8 | 16 | >16 | | | | |
| | | | | | | | 0.5 | | 2 | | | | | | 0.5-2 | 2 | |
| | MI | ≦.015 | .03 | .06 | .12 | .25 | .5 | 1 | 2 | 4 | 8 | 16 | >16 | | | | |
| | | | | | 1 | 2 | | | | | | | | | 0.12-0.25 | .25 | |
| | LZD | | .03 | .06 | .12 | .25 | .5 | 1 | 2 | 4 | 8 | 16 | 32 | >32 | | | |
| | | | | | | | | 2 | 1 | | | | | | 1-2 | 1 | |
| | VA | | .03 | .06 | .12 | .25 | .5 | 1 | 2 | 4 | 8 | 16 | 32 | >32 | | | |
| | | | | | | | | | 1 | 2 | | | | | 1-4 | 4 | |
| | OX | | .03 | .06 | .12 | .25 | .5 | 1 | 2 | 4 | 8 | 16 | 32 | >32 | | | |
| | | | | | | | | | | 1 | | | 1 | 1 | 1->32 | 32 | |

¹Drugs: NB = novobiocin, TE = tetracycline, MI = minocycline, LZD = linezolid, VA = vancomycin, OX = oxacillin
²MSSA = VISE should have a vancomycin MIC of 8 ug/ml: These isolates tested at 4 ug/ml, and therefore are unconfirmed vancomycin-intermediate
Staphylococcus epidermidis

MIC Range, MIC_{50,}, and MIC₉₀ Values for Linezolid-Resistant Staphylococcus aureus Isolates

| Organism | | | | | | | | | | | | | | | | | |
|---|---|---|---|---|---|---|---|---|---|---|---|---|---|---|---|---|---|
| Phenotype | | | | | | | | | | | | | | | MIC | | |
| (No. | | | | | | | | | | | | | | | Range | MIC₅₀ | MIC₉₀ |
| Tested) | Drug¹ | | | | | | | | | | | | | | (μg/ml) | (μg/ml) | (μg/ml) |
| LRSA²(3) | NB/ | .04/ | .08/.03 | .15/.06 | .312/.12 | .625/.25 | 1.25/.5 | 2.5/ | 5/2 | 10/ | 20/ | 40/ | | | | | |
| | TE | .015 | 1 | 2 | | | | 1 | | 4 | 8 | 16 | | | .08/.03-.15/ | .15/.06 | |
| | | | | | | | | | | | | | | | .06 | | |
| | NB/ | .04/ | .08/.03 | .15/.06 | .312/.12 | .625/.25 | 1.25/.5 | 2.5/ | 5/2 | 10/ | 20/ | 40/ | | | | | |
| | MI | .015 | 1 | 2 | | | | 1 | | 4 | 8 | 16 | | | .08/.03-.15/ | .15/.06 | |
| | | | | | | | | | | | | | | | .06 | | |
| | NB | ≦.04 | .08 | .15 | .312 | .62 | 1.25 | 2.5 | 5 | 10 | 20 | 40 | | | | | |
| | | | | 3 | | | | | | | | | | | 0.15 | 0.15 | |
| | TE | .015 | .03 | .06 | .12 | .25 | .5 | 1 | 2 | 4 | 8 | 16 | >16 | | | | |
| | | | | | | | 2 | | | | | | 1 | | 0.5->16 | 0.5 | |
| | MI | .015 | .03 | .06 | .12 | .25 | .5 | 1 | 2 | 4 | 8 | 16 | >16 | | | | |
| | | | | | 2 | | | | 1 | | | | | | 0.12-2 | 0.12 | |
| | LZD | | .03 | .06 | .12 | .25 | .5 | 1 | 2 | 4 | 8 | 16 | 32 | >32 | | | |
| | | | | | | | | | | | | 1 | 1 | 1 | 16->32 | 32 | |
| | VA | | .03 | .06 | .12 | .25 | .5 | 1 | 2 | 4 | 8 | 16 | 32 | >32 | | | |
| | | | | | | | 1 | 2 | | | | | | | 0.5-1 | 1 | |
| | OX | | .03 | .06 | .12 | .25 | .5 | 1 | 2 | 4 | 8 | 16 | 32 | >32 | | | |
| | | | | | | | | | | | | | | 3 | >32 | >32 | |

¹Drugs: NB = novobiocin, TE = tetracycline, MI = minocycline, LZD = linezolid, VA = vancomycin, OX = oxacillin
²LRSA = linezolid-resistant Staphylococcus aureus

APPENDIX A
Line Listing of Minimal Inhibitory Concentration (μg/mL) Values

| | | Micromyx | Minimal Inhibitory Concentration (μg/mL) | | | | | | | | |
|---|---|---|---|---|---|---|---|---|---|---|---|
| Organism | Phenotype | Isolate # | NB/TE¹ | NB² | TE³ | NB/MI⁴ | MI⁵ | LZD⁶ | VA⁷ | OX⁹ | Date |
| Staphylococcus aureus | MSSA⁹ | 753 | 0.15/0.06 | 0.31 | 0.5 | 0.15/0.06 | 0.12 | 2 | 0.5 | 0.25 | 22FEB06 |
| | | 754 | 0.31/0.12 | 0.31 | 0.5 | 0.15/0.06 | 0.12 | 4 | 0.5 | 0.25 | 22FEB06 |
| | | 755 | 0.31/0.12 | 0.31 | 0.5 | 0.15/0.06 | 0.12 | 2 | 1 | 0.25 | 22FEB06 |
| | | 779 | 0.15/0.06 | 0.31 | 0.5 | 0.15/0.06 | 0.12 | 2 | 0.5 | 0.12 | 22FEB06 |
| | | 782 | 0.15/0.06 | 0.15 | 1 | 0.15/0.06 | 0.12 | 2 | 1 | 0.25 | 22FEB06 |
| | | 783 | 0.15/0.06 | 0.31 | 0.5 | 0.15/0.06 | 0.12 | 2 | 1 | 0.25 | 22FEB06 |
| | | 784 | 0.31/0.12 | 0.31 | 16 | 0.15/0.06 | 0.12 | 2 | 0.5 | 0.25 | 22FEB06 |
| | | 785 | 0.15/0.06 | 0.15 | 0.5 | 0.15/0.06 | 0.12 | 2 | 0.5 | 0.12 | 22FEB06 |
| | | 786 | 0.15/0.06 | 0.15 | 0.5 | 0.15/0.06 | 0.12 | 4 | 0.5 | 0.25 | 22FEB06 |
| | | 787 | 0.08/0.03 | 0.15 | 0.5 | 0.08/0.03 | 0.12 | 2 | 0.5 | 0.25 | 22FEB06 |
| | | 788 | 0.15/0.06 | 0.15 | 0.5 | 0.08/0.03 | 0.12 | 2 | 0.5 | 0.25 | 22FEB06 |
| | | 994 | 0.15/0.06 | 0.15 | 0.5 | 0.15/0.06 | 0.12 | 2 | 1 | 0.25 | 22FEB06 |
| | | 996 | 0.15/0.06 | 0.31 | 0.5 | 0.15/0.06 | 0.06 | 2 | 0.5 | 0.25 | 22FEB06 |
| | | 997 | 0.15/0.06 | 0.31 | 0.5 | 0.15/0.06 | 0.12 | 2 | 1 | 0.25 | 22FEB06 |
| | | 999 | 0.15/0.06 | 0.15 | >16 | 0.15/0.06 | 0.12 | 2 | 0.5 | 0.25 | 22FEB06 |
| | | 1000 | 0.15/0.06 | 0.15 | 0.5 | 0.15/0.06 | 0.12 | 2 | 1 | 0.25 | 24FEB06 |
| | | 1001 | 0.31/0.12 | 0.31 | 0.5 | 0.15/0.06 | 0.12 | 2 | 1 | 0.25 | 24FEB06 |
| | | 1005 | 0.15/0.06 | 0.08 | 0.25 | 0.15/0.06 | 0.06 | 2 | 1 | 0.25 | 24FEB06 |
| | | 1006 | 0.31/0.12 | 0.31 | 0.5 | 0.15/0.06 | 0.12 | 2 | 1 | 0.25 | 24FEB06 |
| | | 1007 | 0.15/0.06 | 0.31 | 0.5 | 0.15/0.06 | 0.12 | 4 | 1 | 0.25 | 24FEB06 |
| | | 1008 | 0.31/0.12 | 0.625 | 0.5 | 0.15/0.06 | 0.06 | 2 | 0.5 | 0.25 | 24FEB06 |
| | | 1009 | 0.15/0.06 | 0.15 | 0.5 | 0.15/0.06 | 0.12 | 4 | 0.5 | 0.5 | 24FEB06 |
| | | 1131 | 0.15/0.06 | 0.15 | 0.5 | 0.15/0.06 | 0.06 | 2 | 1 | 0.5 | 24FEB06 |
| | | 1133 | 0.15/0.06 | 0.15 | 0.5 | 0.15/0.06 | 0.12 | 2 | 0.5 | 0.5 | 24FEB06 |
| Staphylococcus aureus | MSSA | 1134 | 0.31/0.12 | 0.31 | 0.5 | 0.15/0.06 | 0.12 | 2 | 0.5 | 0.25 | 24FEB06 |
| | | 789 | 0.15/0.06 | 0.31 | 0.5 | 0.15/0.06 | 0.12 | 4 | 1 | 0.25 | 24FEB06 |
| | | 790 | 0.15/0.06 | 0.31 | 0.5 | 0.15/0.06 | 0.12 | 4 | 0.5 | 0.25 | 24FEB06 |
| | | 791 | 0.15/0.06 | 0.15 | 0.5 | 0.15/0.06 | 0.12 | 2 | 0.5 | 0.25 | 24FEB06 |
| | | 793 | 0.31/0.12 | 0.31 | 0.5 | 0.15/0.06 | 0.12 | 4 | 0.5 | 0.25 | 24FEB06 |
| | | 794 | 0.31/0.12 | 0.31 | 0.5 | 0.15/0.06 | 0.12 | 4 | 1 | 0.25 | 24FEB06 |
| | | 1002 | 0.15/0.06 | 0.15 | 0.5 | 0.15/0.06 | 0.12 | 2 | 0.5 | 0.5 | 24FEB06 |
| | | 754 | 0.31/0.12 | 0.31 | 0.5 | 0.15/0.06 | 0.12 | 4 | 0.5 | 0.25 | 24FEB06 |
| | | 1654 | 0.31/0.12 | 0.31 | 0.5 | 0.15/0.06 | 0.12 | 2 | 0.5 | 0.12 | 03MAR06 |
| | | 1656 | 0.15/0.06 | 0.15 | 0.5 | 0.08/0.03 | 0.06 | 2 | 0.5 | 0.25 | 03MAR06 |
| | | 1662 | 0.15/0.06 | 0.15 | 0.5 | 0.08/0.03 | 0.06 | 2 | 0.5 | 0.25 | 03MAR06 |
| | | 1663 | 0.15/0.06 | 0.15 | 1 | 0.08/0.03 | 0.12 | 4 | 1 | 0.25 | 03MAR06 |
| | | 1666 | 0.15/0.06 | 0.31 | 0.5 | 0.08/0.03 | 0.06 | 4 | 0.5 | 0.25 | 03MAR06 |
| | | 1667 | 0.08/0.03 | 0.15 | 1 | 0.08/0.03 | 0.12 | 4 | 1 | 0.25 | 03MAR06 |
| | | 1668 | 0.15/0.06 | 0.15 | 0.5 | 0.08/0.03 | 0.06 | 4 | 0.5 | 0.25 | 03MAR06 |
| | | 1671 | 0.15/0.06 | 0.15 | 0.5 | 0.08/0.03 | 0.06 | 2 | 1 | 0.25 | 03MAR06 |
| | | 1728** | 0.62/0.25 | 10 | 0.25 | 0.15/0.06 | 0.06 | 1 | 1 | 0.25 | 03MAR06 |
| | | **Novobiocin result confirmed in retest on 09MAR06 as shown in line below | | | | | | | | | |
| | | 1728 | 0.312/0.12 | 10 | 0.25 | 0.15/0.06 | 0.06 | 1 | 1 | 0.12 | 09MAR06 |
| | | 756 | 0.15/0.06 | 0.15 | 0.5 | 0.08/0.03 | 0.06 | 2 | 0.5 | 0.25 | 03MAR06 |
| | | 780 | 0.15/0.06 | 0.31 | 0.5 | 0.15/0.06 | 0.12 | 4 | 1 | 0.25 | 03MAR06 |
| | | 781 | 0.15/0.06 | 0.15 | 0.5 | 0.08/0.03 | 0.06 | 2 | 0.5 | 0.25 | 03MAR06 |
| | | 792 | 0.15/0.06 | 0.15 | 0.5 | 0.08/0.03 | 0.06 | 2 | 0.5 | 0.12 | 03MAR06 |
| | | 995 | 0.15/0.06 | 0.31 | 0.5 | 0.08/0.03 | 0.12 | 2 | 1 | 0.12 | 03MAR06 |
| | | 998 | 0.15/0.06 | 0.31 | 0.5 | 0.08/0.03 | 0.06 | 2 | 1 | 0.12 | 03MAR06 |
| | | 1003 | 0.08/0.03 | 0.08 | 0.5 | 0.08/0.03 | 0.06 | 2 | 0.5 | 0.25 | 03MAR06 |
| | | 1132 | 0.15/0.06 | 0.15 | 0.5 | 0.08/0.03 | 0.06 | 2 | 0.5 | 0.12 | 03MAR06 |
| | | 133 | 0.15/0.06 | 0.15 | 0.5 | 0.08/0.03 | 0.06 | 2 | 0.5 | 0.12 | 03MAR06 |
| | | 134 | 0.08/0.03 | 0.08 | 0.5 | ≦0.04/0.015 | 0.06 | 2 | 0.5 | 0.25 | 03MAR06 |
| | | 135 | 0.15/0.06 | 0.31 | 0.5 | 0.08/0.03 | 0.06 | 4 | 1 | 0.25 | 03MAR06 |
| Staphylococcus aureus | MRSA¹⁰ | 763 | 0.08/0.03 | 0.08 | 0.5 | 0.15/0.06 | 0.12 | 4 | 1 | 32 | 22FEB06 |
| | | 765 | 0.15/0.06 | 0.15 | 0.5 | 0.08/0.03 | 0.06 | 4 | 0.5 | >32 | 22FEB06 |
| | | 766 | 0.08/0.03 | 0.08 | 1 | 0.15/0.06 | 0.12 | 4 | 0.5 | >32 | 22FEB06 |
| | | 767 | 0.08/0.03 | 0.15 | 0.5 | 0.08/0.03 | 0.12 | 2 | 0.5 | 16 | 22FEB06 |
| | | 768 | 0.15/0.06 | 0.15 | 0.5 | 0.15/0.06 | 0.06 | 2 | 0.5 | 8 | 22FEB06 |
| | | 769 | 0.08/0.03 | 0.15 | >16 | 0.08/0.03 | 0.12 | 2 | 0.5 | 16 | 22FEB06 |
| | | 770 | ≦0.04/0.015 | ≦0.04 | 0.5 | 0.08/0.03 | 0.12 | 2 | 0.5 | 16 | 22FEB06 |
| | | 771 | 0.08/0.03 | 0.08 | 0.5 | 0.15/0.06 | 0.12 | 2 | 1 | >32 | 22FEB06 |
| | | 772 | 0.15/0.06 | 0.15 | 0.5 | 0.15/0.06 | 0.12 | 2 | 0.5 | 32 | 22FEB06 |
| | | 1010 | 0.15/0.06 | 0.31 | 4 | 0.15/0.06 | 0.12 | 2 | 0.5 | 32 | 22FEB06 |
| | | 1012 | 0.31/0.12 | 0.31 | 1 | 0.15/0.06 | 0.12 | 2 | 0.5 | >32 | 22FEB06 |
| | | 1013 | 0.15/0.06 | 0.31 | 0.5 | 0.15/0.06 | 0.12 | 2 | 0.5 | 32 | 22FEB06 |
| | | 1014 | 0.15/0.06 | 0.15 | 0.5 | 0.15/0.06 | 0.12 | 2 | 0.5 | 32 | 22FEB06 |
| | | 1015 | 0.08/0.03 | 0.08 | 0.5 | 0.08/0.03 | 0.12 | 2 | 0.5 | 32 | 22FEB06 |
| | | 1016 | 0.15/0.06 | 0.31 | 0.5 | 0.15/0.06 | 0.12 | 2 | 0.5 | 8 | 22FEB06 |
| | | 1017 | 0.15/0.06 | 0.08 | 0.5 | 0.15/0.06 | 0.12 | 2 | 0.5 | 16 | 22FEB06 |
| | | 1021 | 0.15/0.06 | 0.15 | 0.5 | 0.15/0.06 | 0.12 | 2 | 0.5 | 32 | 22FEB06 |
| | | 1022 | 0.15/0.06 | 0.31 | 0.5 | 0.15/0.06 | 0.12 | 2 | 0.5 | 8 | 22FEB06 |
| | | 1023 | 0.15/0.06 | 0.15 | 0.5 | 0.15/0.06 | 0.12 | 2 | 0.5 | 4 | 22FEB06 |
| | | 1024 | 0.15/0.06 | 0.15 | 0.5 | 0.15/0.06 | 0.12 | 2 | 0.5 | 16 | 22FEB06 |
| | | 1025 | 0.15/0.06 | 0.15 | 0.5 | 0.15/0.06 | 0.12 | 2 | 1 | 16 | 22FEB06 |
| | | 1135 | 0.15/0.06 | 0.31 | 0.5 | 0.15/0.06 | 0.12 | 2 | 0.5 | 32 | 22FEB06 |
| | | 1136 | 0.15/0.06 | 0.31 | 0.5 | 0.15/0.06 | 0.12 | 4 | 0.5 | 32 | 22FEB06 |
| | | 1137 | 0.15/0.06 | 0.62 | 0.5 | 0.15/0.06 | 0.12 | 2 | 0.5 | >32 | 22FEB06 |
| | | 1138 | 0.15/0.06 | 0.15 | 1 | 0.15/0.06 | 0.12 | 4 | 0.5 | 8 | 22FEB06 |
| | | 1004 | 0.31/0.12 | 0.31 | 16 | 0.15/0.06 | 0.12 | 4 | 1 | 8 | 24FEB06 |
| | | 757 | 0.31/0.12 | 0.31 | >16 | 0.15/0.06 | 8 | 4 | 1 | 32 | 24FEB06 |
| | | 773 | 0.15/0.06 | 0.31 | 0.5 | 0.15/0.06 | 0.12 | 2 | 0.5 | 8 | 24FEB06 |
| | | 774 | 0.15/0.06 | 0.31 | 0.5 | 0.15/0.06 | 0.12 | 2 | 1 | 32 | 24FEB06 |
| | | 775 | 0.15/0.06 | 0.15 | 0.5 | 0.15/0.06 | 0.12 | 4 | 1 | >32 | 24FEB06 |
| | | 776 | 0.15/0.06 | 0.15 | 0.5 | 0.15/0.06 | 0.12 | 2 | 1 | >32 | 24FEB06 |
| | | 777 | ≦0.04/0.015 | ≦0.04 | 0.5 | 0.08/0.03 | 0.12 | 2 | 0.5 | 32 | 24FEB06 |
| | | 778 | <0.04/0.015 | ≦0.04 | 0.25 | 0.08/0.03 | 0.06 | 2 | 1 | >32 | 24FEB06 |
| | | 886 | 0.15/0.06 | 0.15 | 0.5 | 0.08/0.03 | 0.12 | 2 | 1 | 32 | 24FEB06 |
| | | 887 | 0.08/0.03 | 0.08 | 0.5 | 0.15/0.06 | 0.12 | 4 | 0.5 | >32 | 24FEB06 |
| | | 888 | 0.15/0.06 | 0.15 | 0.5 | 0.15/0.06 | 0.12 | 4 | 1 | >32 | 24FEB06 |
| | | 889 | 0.15/0.06 | 0.15 | 1 | 0.15/0.06 | 0.12 | 4 | 1 | >32 | 24FEB06 |
| | | 1222 | 0.15/0.06 | 0.08 | 0.5 | 0.08/0.03 | 0.12 | 2 | 1 | 32 | 24FEB06 |
| | | 758 | 0.08/0.03 | 0.15 | 0.5 | 0.15/0.06 | 0.12 | 4 | 1 | >32 | 24FEB06 |
| | | 760 | 0.15/0.06 | 0.15 | 0.5 | 0.15/0.06 | 0.12 | 4 | 1 | >32 | 24FEB06 |
| | | 1730 | 0.15/0.06 | 0.15 | 0.5 | 0.15/0.06 | 0.12 | 4 | 1 | 32 | 24FEB06 |
| | | 2009 | ≦0.04/0.015 | ≦0.04 | >16 | ≦0.04/0.015 | 1 | 2 | 2 | >32 | 24FEB06 |
| | | 2010 | 0.15/0.06 | 0.15 | 0.5 | 0.15/0.06 | 0.12 | 2 | 0.5 | 16 | 24FEB06 |
| | | 2011 | 0.15/0.06 | 0.15 | >16 | 0.15/0.06 | 0.12 | 4 | 1 | 32 | 24FEB06 |
| | | 1729 | 0.08/0.03 | 0.08 | >16 | 0.08/0.03 | 16 | 2 | 2 | >32 | 03MAR06 |
| | | 1222 | 0.08/0.03 | 0.08 | 0.5 | 0.08/0.03 | 0.06 | 2 | 1 | 32 | 03MAR06 |
| | | 1653 | ≦0.04/0.015 | ≦0.04 | 0.25 | ≦0.04/0.015 | 0.06 | 2 | 1 | >32 | 03MAR06 |
| | | 1658 | 0.08/0.03 | 0.08 | 0.5 | ≦0.04/0.015 | 0.06 | 2 | 0.5 | 16 | 03MAR06 |
| | | 1659 | 0.08/0.03 | 0.08 | 0.25 | 0.08/0.03 | 0.06 | 2 | 1 | 16 | 03MAR06 |
| | | 1661 | ≦0.04/0.015 | ≦0.04 | 0.5 | ≦0.04/0.015 | 0.12 | 4 | 1 | >32 | 03MAR06 |
| Staphylococcus aureus | MRSA | 758 | ≦0.04/0.015 | 0.08 | 1 | 0.08/0.03 | 0.12 | 4 | 1 | >32 | 03MAR06 |
| | | 760 | 0.08/0.03 | 0.08 | 1 | 0.08/0.03 | 0.12 | 2 | 1 | >32 | 03MAR06 |
| Staphylococcus aureus | VISA¹¹ | 2012 | 0.08/0.03 | 0.08 | 2 | 0.08/0.03 | 0.25 | 1 | 8 | >32 | 03MAR06 |
| | | 2018 | ≦0.04/0.015 | ≦0.04 | 0.25 | ≦0.04/0.015 | 0.06 | 2 | 8 | >32 | 03MAR06 |
| | Unconfirmed | 2013 | 0.15/0.06 | 0.08 | 0.5 | 0.08/0.03 | 0.12 | 2 | 4 | 4 | 03MAR06 |
| | Unconfirmed | 2014 | 0.15/0.06 | 0.15 | >16 | 0.15/0.06 | 2 | 2 | 4 | >32 | 03MAR06 |
| | Unconfirmed | 2015 | ≦0.04/0.015 | ≦0.04 | 0.06 | ≦0.04/0.015 | <0.015 | 1 | 4 | >32 | 03MAR06 |
| | Unconfirmed | 2016 | ≦0.04/0.015 | ≦0.04 | 0.5 | 0.08/0.03 | 0.06 | 4 | 4 | 4 | 03MAR06 |
| | Unconfirmed | 2017 | 0.15/0.06 | 0.15 | 0.25 | 0.08/0.03 | 0.03 | 2 | 4 | 0.25 | 03MAR06 |
| | Unconfirmed | 2019 | ≦0.04/0.015 | ≦0.04 | >16 | ≦0.04/0.015 | 2 | 1 | 4 | >32 | 03MAR06 |
| Staphylococcus | VISE¹² | 2022 | ≦0.04/0.015 | ≦0.04 | 2 | 0.08/0.03 | 0.25 | 2 | 8 | 16 | 03MAR06 |
| epidermidis | | 2023 | ≦0.04/0.015 | <0.04 | 0.5 | 0.08/0.03 | 0.06 | 2 | 8 | 2 | 03MAR06 |
| | | 2020 | 0.08/0.03 | 0.08 | >16 | 0.08/0.03 | 0.12 | 2 | 8 | 8 | 09MAR06 |
| | | 2021 | ≦0.04/0.015 | ≦0.04 | 0.25 | ≦0.04/0.015 | 0.06 | 1 | 8 | 0.5 | 09MAR06 |
| | Unconfirmed | 2026 | ≦0.04/0.015 | ≦0.04 | 2 | 0.08/0.03 | 0.25 | 2 | 4 | 32 | 03MAR06 |
| | Unconfirmed | 2024 | 5/2 | 5 | 2 | 0.625/0.25 | 0.25 | 1 | 2 | >32 | 09MAR06 |
| | Unconfirmed | 2025 | 0.312/0.12 | 0.312 | 0.5 | 0.15/0.06 | 0.12 | 1 | 4 | 4 | 09MAR06 |
| Staphylococcus aureus | LRSA¹³ | 1651 | 0.08/0.03 | 0.15 | 0.5 | 0.08/0.03 | 0.12 | 16 | 0.5 | >32 | 24FEB06 |
| | | 1652 | 0.15/0.06 | 0.15 | 0.5 | 0.15/0.06 | 0.12 | 32 | 1 | >32 | 24FEB06 |
| | | 1725 | 0.15/0.06 | 0.15 | >16 | 0.15/0.06 | 2 | >32 | 1 | >32 | 24FEB06 |

¹Novobiocin/Tetracycline.
²Novobiocin
³Tetracycline
⁴Novobiocin/Minocycline
⁵Minocycline
⁶Linezolid
⁷Vancomycin
⁸Oxacillin
⁹Methicillin-susceptible Staphylococcus aureus
¹⁰Methicillin-resistant Staphylococcus aureus
¹¹Vancomycin intermediate Staphylococcus aureus
¹²Vancomycin intermediate Staphylococcus epidermidis
¹³Linezolid-resistant Staphylococcus aureus

APPENDIX B
Quality Control Results

| | | Minimal Inhibitory Concentration (μg/mL) | | | | | | | | |
|---|---|---|---|---|---|---|---|---|---|---|
| Organisim | ATCC¹ No. | NB/TE² | NB³ | TE⁴ | NB/MI⁵ | MI⁶ | LZD⁷ | VA⁸ | OX⁹ | Date |
| Staphylococcus aureus | 29213 | 0.15/0.06 | 0.15 | 0.5 | 0.15/0.06 | 0.12 | 4 | 0.5 | 0.12 | 22FEB06 |
| | | 0.15/0.06 | 0.31 | 1 | 0.15/0.06 | 0.12 | 4 | 1 | 0.12 | 22FEB06 |
| | | 0.15/0.06 | 0.15 | 0.5 | 0.15/0.06 | 0.12 | 4 | 0.5 | 0.12 | 22FEB06 |
| | 29213 | 0.15/0.06 | 0.15 | 0.5 | 0.15/0.06 | 0.12 | 4 | 0.5 | 0.25 | 24FEB06 |
| | | 0.15/0.06 | 0.15 | 0.5 | 0.15/0.06 | 0.12 | 4 | 0.5 | 0.25 | 24FEB06 |
| | | 0.15/0.06 | 0.15 | 0.5 | 0.15/0.06 | 0.12 | 4 | 1 | 0.25 | 24FEB06 |
| | 29213 | 0.15/0.06 | 0.15 | 0.5 | 0.08/0.03 | 0.06 | 4 | 0.5 | 0.12 | 03MAR06 |
| | | 0.15/0.06 | 0.15 | 0.5 | 0.08/0.03 | 0.12 | 4 | 0.5 | 0.12 | 03MAR06 |
| | | 0.15/0.06 | 0.15 | 0.5 | 0.08/0.03 | 0.06 | 4 | 0.5 | 0.12 | 03MAR06 |
| | 29213 | 0.15/0.06 | 0.15 | 0.5 | 0.08/0.03 | 0.06 | 2 | 0.5 | 0.12 | 09MAR06 |
| CLSI¹⁰ QC Range | | NA¹¹ | NA | 0.12-1 | NA | 0.06-0.5 | 1-4 | 0.5-2 | 0.12-0.5 | |
| Percent Values in Range | | | | 100 | | 100 | 100 | 100 | 100 | |
| Enterococcus faecalis | 29212 | 5/2 | 5 | 16 | 2.5/1 | 2 | 2 | 2 | 8 | 22FEB06 |
| | | 5/2 | 5 | 16 | 2.5/1 | 2 | 2 | 2 | 8 | 22FEB06 |
| | | 5/2 | 5 | 16 | 2.5/1 | 2 | 2 | 2 | 8 | 22FEB06 |
| | 29212 | 5/2 | 5 | 16 | 2.5/1 | 2 | 2 | 2 | 8 | 24FEB06 |
| | | 5/2 | 5 | 16 | 2.5/1 | 2 | 2 | 2 | 8 | 24FEB06 |
| | | 5/2 | 5 | 16 | 2.5/1 | 2 | 2 | 4 | 8 | 24FEB06 |
| | 29212 | 2.5/1 | 5 | 16 | 2.5/1 | 1 | 2 | 2 | 8 | 03MAR06 |
| | | 5/2 | 5 | 16 | 2.5/1 | 2 | 2 | 2 | 8 | 03MAR06 |
| | | 25-1 | 5 | 16 | 2.5/1 | 2 | 2 | 2 | 8 | 03MAR06 |
| | 29212 | 5/2 | 5 | 16 | 2.5/1 | 2 | 2 | 2 | 8 | 09MAR06 |
| CLSI QC Range | | NA | NA | 8-32 | NA | 1-4 | 1-4 | 1-4 | 8-32 | |
| Percent Values in Range | | | | 100 | | 100 | 100 | 100 | 100 | |

¹American Type Culture Collection
²Novobiocin/Tetracycline
³Novobiocine
⁴Tetracycline
⁵Novobiocin/Minocycline
⁶Minocycline
⁷Linezolid
⁸Vancomycin
⁹Oxacillin
¹⁰Clinical and Laboratory Standards Institute
¹¹Not applicable

The following is an example that illustrates a procedure for practicing the invention. This example should not be construed as limiting.

### EXAMPLE 1

In the illustrated embodiment, a 250 mg per dose of tetracycline is combined with a 250 mg dose of novobiocin. The combination can be given two to four times daily orally for 5-14 days. This combination should treat suspected Gram-positive infections, such as skin and soft tissue infections, urinary tract infections, sinusitis, bronchitis, or pneumonia.

The methods of administering the combination can also include, for example, the use of the pharmaceutical combination in an antibiotic ointment or in an opthalmologic formulation as eye drops or ocular ointment. Formulations of oral liquid or suspension for pediatric or geriatric use and formulation as a sterile solution for intravenous administration.

The above description is considered that of the preferred embodiments only. Modifications of the invention will occur to those skilled in the art and to those who make or use the invention. Therefore, it is understood that the embodiments described above are merely for illustrative purposes and not intended to limit the scope of the invention.

1. A method for treating an infection from Gram-positive bacteria, comprising:
administering to a human subject a composition comprising a combination of a gyrase B inhibitor and a protein synthesis inhibitor in an amount effective to reduce or eliminate the Gram-positive bacterial infection.

2. The method according to claim 1, wherein the Gram-positive bacteria is Staphylococcus aureus.

3. The method according to claim 1, wherein the Gram-positive bacteria is methicill in-resistant Staphylococcus aureus.

4. The method according to claim 1, wherein the Gram-positive bacteria is community acquired methicillin-resistant Staphylococcus aureus.

5. The method according to claim 1, wherein the composition is administered in a solid oral dosage form.

6. The method according to claim 5, wherein said dosage form is selected from a group consisting of tablets, pills, caplets, and capsules.

7. The method according to claim 1, wherein the gyrase B inhibitor is novobiocin.

8. The method according to claim 1, wherein the protein synthesis inhibitor is a tetracycline.

9. The method according to claim 8, wherein the protein tetracycline is minocycline.

10. The method according to claim 8, wherein the protein tetracycline is doxycycline.

11. A method of treating a methicillin-resistant Staphylococcus aureus infection, comprising:
administering to a human subject a composition comprising a combination of a gyrase B inhibitor and a protein synthesis inhibitor in an amount effective to reduce or eliminate the methicillin-resistant Staphylococcus aureus infection.

12. The method of claim 11, wherein the gyrase B inhibitor is novobiocin.

13. The method of claim 11, wherein the protein synthesis inhibitor is minocycline.

14. A composition for the treatment of Gram-positive bacterial infections, comprising:
a combination of a gyrase B inhibitor and minocycline.

15. The composition of claim 14, wherein the gyrase B inhibitor is novobiocin.

16. The composition of claim 14, wherein the Gram-positive bacterial infection is methicill in-resistant Staphylococcus aureus.
